(19) United States
(12) Patent Application Publication
(10) Pub. No.: US 2024/0382118 A1
(43) Pub. Date: Nov. 21, 2024

(54) Shelf-Stable Sterilization of Aptamer-Sensors for In-Vivo Measurement in Humans

(71) Applicant: University of Cincinnati, Cincinnati, OH (US)

(72) Inventor: Jason Heikenfeld, Cincinnati, OH (US)

(73) Assignee: University of Cincinnati, Cincinnati, OH (US)

(21) Appl. No.: 18/690,025

(22) PCT Filed: Sep. 7, 2022
(86) PCT No.: PCT/US22/42769

§ 371 Date: Mar. 7, 2024

Related U.S. Application Data
(60) Provisional application No. 63/241,211, filed on Sep. 7, 2021.
(60) Provisional application No. 63/333,767, filed on Apr. 22, 2022.

(51) Int. Cl. A61B 5/145; A61B 5/1473

(52) U.S. Cl. CPC A61B 5/14546; A61B 5/14532; A61B 5/1473

(57) ABSTRACT

A method of measuring analytes in a subject in vivo for a period of time is provided. The method involves storing a device. The device includes at least one sensor comprising an aptamer material; at least one feature for coupling said sensor to an analyte in the subject in vivo; at least one sterilization state that imparts sterilization on at least one component of the device; at least one sterile packaging material enabling a storage state; and at least one aptamer storage material. The sensor is contained in the sterile packaging material and the storage material is anhydrous. Further, wherein the sensor and the feature are sterile. The method further involves removing the sterile packaging material from the device and using the feature to couple the sensor to an analyte in the subject.

### CROSS-REFERENCE TO RELATED APPLICATIONS

This application is a continuation of PCT Application No. PCT/US22/42769 filed Sep. 7, 2022, which claims the benefit of the filing date of U.S. Provisional Application No. 63/241,211 filed Sep. 7, 2021, and U.S. Provisional Application No. 63/333,767 filed Apr. 22, 2022, the disclosures of which are incorporated by reference herein in their entireties.

### TECHNICAL FIELD

The present invention relates to the use of electrochemical, aptamer-based (E-AB) sensors.

### BACKGROUND OF THE INVENTION

Electrochemical aptamer sensors consist of an aptamer sequence that specifically binds to an analyte of interest, that along with a blocking layer are both incubated onto a sensing monolayer to an electrode, and the aptamer having an attached redox active molecule (redox tag) which can transfer electrical charge to or from the electrode. When an analyte binds to the aptamer, the aptamer changes shape, changing the availability for electron transfer between the redox tag and the electrode, resulting in a measurable change in electrical current that can be translated to a measure of concentration of the analyte. Aptamer sensors can also be optical based, using molecular beacon type aptamers where analyte binding changes a measurable fluorescence from an optical tag on the aptamer. Aptamer sensors have largely been relegated to applications where sterilization is not a major issue, such as a laboratory assay which are ex-vivo (fluids collected from the body but aptamers are never placed into the body), or for example in-vivo animal testing where the sensor is simply disinfected very near the time of usage (hours, days at maximum) and is not stored sterile for prolonged periods of time (weeks, months). Therefore, proper shelf-stable sterilization of aptamers sensors has not needed to be addressed, and if aptamer sensors are adapted to commercial in-vivo human use, via microneedles, indwelling needles, or even implantable sensors, then shelf-stable sterilization techniques must be developed and implemented for aptamer sensors.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Embodiments of the disclosed invention are directed to aptamer sensors that can be used in vivo for extended periods of time after being stored in a sterile state.

And so, one aspect of the present invention is directed to an aptamer-based device. The device is useful for measuring analytes in a subject in vivo for a period of time. The device includes at least one sensor comprising an aptamer material; at least one feature for coupling said sensor to an analyte in the subject in vivo; at least one sterilization state that imparts sterilization on at least one component of the device; at least one sterile packaging material enabling a storage state; and at least one aptamer storage material. The sensor is contained in the sterile packaging material and the storage material is anhydrous. Further, wherein the sensor and the feature are sterile.

### BRIEF DESCRIPTION OF THE DRAWINGS

The objects and advantages of the disclosed invention will be further appreciated in light of the following detailed descriptions and drawings in which:

FIG. 1 is a schematic of an embodiment of the present invention.

FIG. 2 is a schematic of an embodiment of the present invention.

FIG. 3 is a schematic of an embodiment of the present invention.

FIG. 4 is a schematic of an embodiment of the present invention.

### DEFINITIONS

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function. Aptamers can be optical or electrochemically detectable in nature using attached fluorescent and optical tags and quenchers as used in molecular beacons (e.g. 6-FAM, HEX, Cyanine, BHQ, DABCYL, etc.), or for example methylene blue redox molecule tagged as is used in electrochemical aptamer-based sensors. Aptamers can function as a single nucleotide strand or as two or more strands whose binding to each other is changed in the presence of an analyte to be measured.

The devices and methods described herein encompass the use of sensors. A “sensor”, as used herein, is a device that is capable of measuring the concentration of a target analyte in solution. As used herein, an “analyte” may be any inorganic or organic molecule, for example: a small molecule drug, a metabolite, a hormone, a peptide, a protein, a carbohydrate, a nucleic acid, or any other composition of matter. The target analyte may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

As used herein, the term “continuous sensing” simply means the device records a plurality of readings over time. Even a point-of-care testing device which provides a single data point can be considered a continuous sensing device if, for example, it is a 15 minute test, that operates by taking multiple data points over 15 minutes and averaging them to provide a single data measure.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “aptamer material” means the portion of a continuous sensing aptamer device that contains aptamer that responds to changes in analyte concentration and which therefore provides a measurement of that analyte. Aptamer material could be for example, a monolayer of aptamer thiol-bonded or non-thiol-bonded to a gold working electrode used for electrochemical measurements. Examples of this technique are found in PCT/US21/51869, filed Sep. 24, 2021, and entitled “Highly Chemically Stable Aptamer Sensors,” the contents of which is incorporated by reference herein in its entirety. Aptamer material could be for example, a solution with molecular-beacon aptamers, examples of which are found in U.S. provisional application 63/136,262, filed Jan. 12, 2021, and entitled “Continuous Optical Aptamer Sensors,” the contents of which is incorporated by reference herein in its entirety. or a solution with redox-tagged aptamers near an electrode, examples of which are found in U.S. provisional application 63/085,484, filed Sep. 30, 2020, and entitled “Solute-Phase Electrochemical Aptamer Sensors for Improved Longevity and Sensitivity,” the contents of which is incorporated by reference herein in its entirety. Aptamer material could be for example, bound to a solid material. Examples of this technique are found in U.S. provisional application 63/122,003, filed Dec. 7, 2020, and entitled “Electrochemical Aptamer Sensors with Aptamers Bound Adjacent to the Electrode,” the contents of which is incorporated by reference herein in its entirety. Other possibilities are possible as well, as long at the aptamer provides a measurement of the analyte as described herein. With sterilization, aptamer material will often be the most sensitive material in the system to degradation by sterilization.

As used herein, the term “aptamer storage material” means a material such as buffered water, water ethanol mixture, glycerin, dry sugar such as trehalose, inert gas such as nitrogen, or other suitable material including any additives used to preserve the aptamer material during shelf storage.

As used herein, the term “sterile packaging material” means the material used to enclose the entire sensing device or the portion of the device (such as a needle) that is inserted into the body, where the sterile packaging material keeps in-vivo exposed portion of the device sterile during shelf storage.

As used herein, the term “device material” means the rest of the device outside of the sensor unit (electronics, housing, skin adhesives, etc.) and may or may not require sterilization as well. For example, with an implanted device, the entire device material must be sterilized, but that is not the case necessarily for a wearable device that simply places a needle-based sensor into the skin.

As used herein, the term “sterilization method” means any suitable method to sterilize a device that meets regulatory standards for use of the device with humans. Such standards may vary from application to region, and therefore are only as definite as required to meet such standards required for use of the sensing device.

As used herein, the term “sensor signal loss due to sterilization” means the sterilization-induced loss in magnitude of measured signal that provides a measurement of the analyte. Sensor signal loss is measurable by simply test the sensor before and after sterilization.

As used herein, the term “storage state or period” means the period between sterilization and human in-vivo use of the sensing device. Storage periods typically at minimum are weeks and commercially typically are multiple months or more due to supply chain economics and timing.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. For example, aptamer sensors typically require separate working, counter, and reference electrodes, but the present invention simply focuses its discussion on the working electrode which contains the sensing transducing element in the form of a monolayer of aptamers.

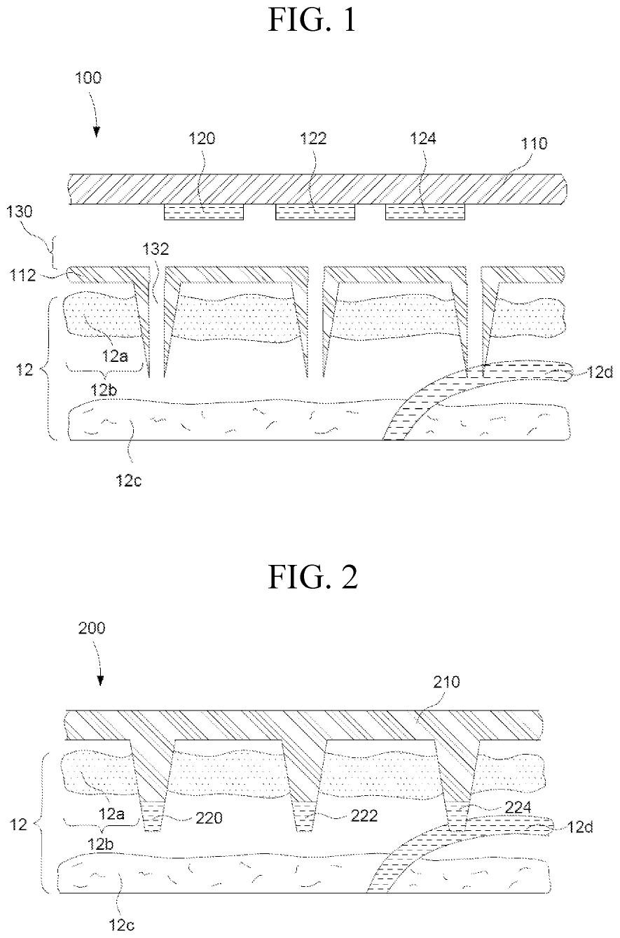

With reference to FIG. 1, in an embodiment of the present invention, a device 100 is placed partially in-vivo into the skin 12 comprised of the epidermis 12a, dermis 12b, and the subcutaneous or hypodermis 12c. A portion of the device 100 accesses fluids such as interstitial fluid from the dermis 12b and/or blood from a capillary 12d. Access is provided, for example, by microneedles 112 formed of metal, polymer, semiconductor, glass or other suitable material, and may include a hollow lumen 132 that contributes to a sample volume. Sample volume is also contributed to by volume 130 above material from which the microneedles 112 project below to electrodes or sensors 120, 122, 124 on polymer substrate 110. Together the volume of volume 130 and lumen 132 form a sample volume and can be a microfluidic component such as channels, a hydrogel, or other suitable material. A diffusion and/or advective flow pathway exists from the invasive biofluid such as interstitial fluid or blood to electrodes or sensors 120, 122, 124, the pathway beginning at the inlet to the microneedle 112, first reaching the electrodes or sensors 120, 122, 124. Alternative arrangements and materials are possible, such as using a single needle, hydrogel polymer microneedles, or other suitable means to couple an invasive fluid to one or more sensors, although these alternative arrangements and materials are not explicitly shown in the figures. In addition, one or more of the features of device 100 or the entire device 100 may be implanted into the body and perform similarly as described herein.

With further reference to FIG. 1, electrodes or sensors 120, 122, 124 may be an affinity-based electrochemical aptamer sensor that has a redox tag such as methylene blue. At least one electrode should be a working electrode functionalized with aptamer and mercaptohexanol blocking layer and one or more of the other electrodes may be counter and or reference potential electrodes. Sensors 120, 122, 124 could also be optical or other forms of aptamer sensors such as impedimetric. Even though sensors 120, 122, 124 are ex-vivo, they are coupled to the body through the epidermis 12a and part of a device that forms an in-vivo measurement and which therefore requires shelf-stable sterilization. Sensors 120, 122, 124 or other features of device 100 may also require additional materials such as membrane to keep cellular content away from sensors, or to retain aptamers in solution near an optical sensor or an working electrode, and such additional materials can be assumed to need to be sterilized as well.

With reference to FIG. 2, where like numerals refer to like features, device 200 includes a polymer substrate 210 and sensors and electrodes 220, 222, 224, that are located in the dermis 12b.

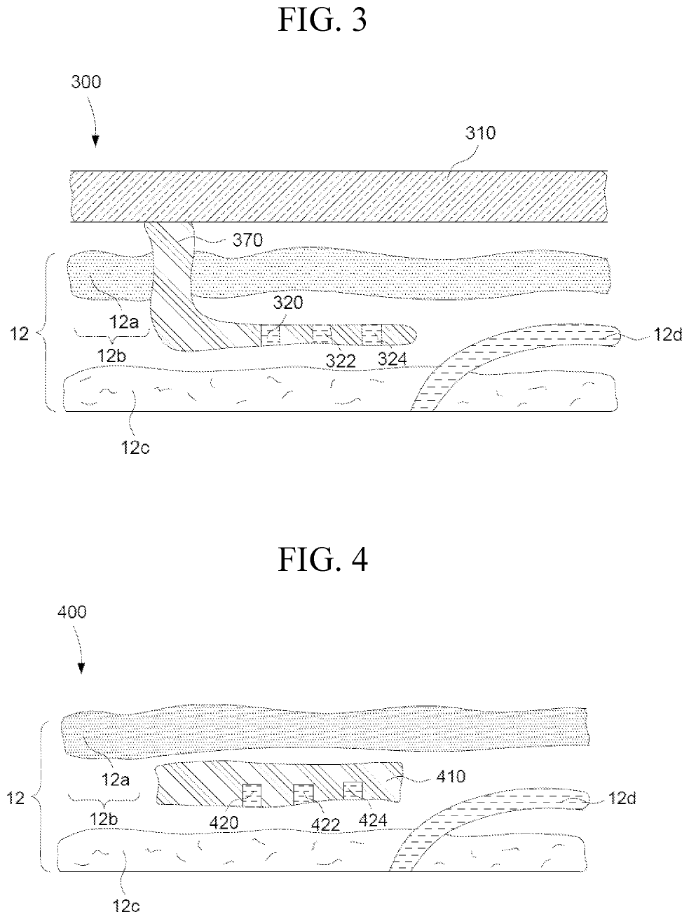

With reference to FIG. 3, where like numerals refer to like features, device 300 includes a polymer substrate 310 and a needle 370 that inserts sensors and electrodes 320, 322, 324 in the dermis.

With reference to FIG. 4 where, like numerals refer to like features, device 400 includes an implanted device where the entire device is in the skin 12 or some other location in the body and comprises a polymer substrate 410 carrying sensors and electrodes 420, 422, 424.

With reference to FIGS. 1-4 and embodiments of the present invention, a device comprises at least one sensor that at least in part comprises an aptamer material, to continuously detect an analyte in the human body. Embodiments of the present invention may further comprise an aptamer storage material, that enables storage of the sensor and/or device for a storage period. Embodiments of the present invention employ a sterilization method that is described in greater detail herein.

With reference to embodiments of the present invention, sterilization methods may include methods ranging from autoclaving to irradiation to other methods, which respectively can result in sensor signal losses due to sterilization that are >90% or less than 10%, or are negligible or immeasurable. For example, an aptamer sensor with a 200% signal gain with analyte binding could endure 20× signal loss due to sterilization and still have a 10% signal gain to analyte binding, which would be above the noise/background changes in signal that are less than 5%. Some sensors will have much weaker signal gains or large noise/background signals, or interference signals, such that sensor signal loss due to sterilization must be very small (e.g. <20%). Sensor signal loss due to sterilization may be, in alternative embodiments, less than 80%, 40%, 20%, 10% or 5%.

With reference to embodiments of the present invention, for factory-calibrated devices the signal gain is calibrated at the factory such that the end-user does not need to calibrate the device with a blood draw analysis or other suitable technique. Signal gain for aptamer sensors is change in signal caused by increase or decrease in target analyte. There are other measures as well for aptamer sensors beyond signal gain, such as chronoampermetrically which while not provided in an exhaustive list are included within the spirit of a factory-calibrated device. Factory calibration presents a challenge, because the device typically cannot be easily measured again after sterilization without compromising sterilization, and therefore the sterilization changes the calibrated signal gain of the sensor. Therefore the present invention includes a factory calibrated signal that changes by less than 80%, 40%, 20%, 10%, or 5%, and the device may remain sterile and shelf stable, in alternative embodiments, for at least 2 weeks, 2 months, or 6 months.

With reference to embodiments of the present invention, by using a reduced scanning window a sensor device can have a longevity of at least 3 days, but this is only possible of the aptamer and blocking monolayer is not overly damaged as taught using the methods herein.

With reference to embodiments of the present invention, sterilization may be performed separately on device and device materials and integrated in a sterile environment, at the cost and complexity of doing so. For example, a sensor that is a gold electrode with an aptamer material for vancomycin, a blocking layer of mercaptohexanol, may be sterilized by e-beam or gamma irradiation, an aptamer storage material such as trehalose or denatured serum boiled in water, cooled, coated and dried onto the electrode/aptamer/blocking layer in a sterile environment, the sensor attached to a sterilized manifold that electrically connects it to a reusable reader at the time of use, and the sensor+aptamer storage material+manifold all sealed sterilized in a metal foil pouch with argon inside of it. In one embodiment, a sensor is sterilized chemically then sealed by a sterile membrane that is not transparent to UV light, and the final device or sensing component then sterilized under UV while the aptamer material is protected from the UV light. In this embodiment, a plurality of components are sterilized and then assembled in sterile environment.

With reference to embodiments of the present invention, a device with sensor that has a plurality of optical aptamers as aptamer material or electrochemical aptamers with redox tags as aptamer material, aptamer storage material such as trehalose which is optional, and a manifold, are all assembled and sterilized together using dry heat in a nitrogen or other inert environment at 120 Celsius for 1 to 10 hours. In a sterile environment these sterilized components are then sealed in a sterile pouch that forms a sterile packing material, or can be sterilized in that same pouch. An inert environment may also benefit from sterilization with radiation (e-beam, gamma, etc.) because radiation can cause localized heating of the sensors that could otherwise degrade the sensors in a non-inert environment containing oxygen or water vapor.

In some embodiments of the present invention, packaged sensors or their subcomponents are sterilized using heat. Autoclave sterilization for 30 minutes at 122° C. can damage an electrochemical aptamer sensor by accelerating detachment of the monolayer of aptamer and blocking layer. At high temperatures and long durations, for some forms of electrochemical aptamers their blocking layer is formed of alkythiols on gold working electrodes which can readily thermally desorb and degrade the sensor signal. These blocking molecules therefore need to be ‘frozen in place’ using for example a 10's nm to microns thick coating of trehalose. Among several sugars such as sucrose, trehalose has been known to be a superior stabilizer in providing protection to biological materials against dehydration and desiccation. Dihydrate trehalose has a melting point of 97° C. which can be too low for dry heat sterilization, while anhydrous trehalose has a melting point of 203° C. which is suitable for dry heat sterilization. In one embodiment, an aptamer sensor preserves aptamers in a coating of 1 μm of trehalose that is first vacuum dried at room temperature, and then slowly ramped over 30 minutes to 170°° C. for at least 30 minutes dry sterilization under dry conditions such as vacuum to further drive off water, enabling a dry sterilization with an anhydrous aptamer storage material. In another embodiment, the dry heat sterilization is performed in a dry gas such as nitrogen, argon, or even dry oxygen if the temperature and duration are adequately low enough. Often, the sterilization temperature is proportional to the time. In various embodiments, the sterilization temperature is 170° C. (340° F.) for 30 minutes, 160° C. (320° F.) for 60 minutes, or 150° C. (300° F.) for 150 minutes or longer. In this embodiment, the aptamer sensor and aptamer storage material are sterilized together with an aptamer storage material that is stable during sterilization.

With reference to embodiments of the present invention, in any sterilization method if an aptamer storage material is used ideally it will be thin enough to not fully protect species such as spores or microbes, and spores are typically about 1 μm in length. Therefore, the aptamer storage material may be, in alternate embodiments, at least <500 nm, <100 nm, or <20 nm, in thickness.

With reference to embodiments of the present invention, a device with a sensor containing aptamer material and an entire rest of the device (electronics, housing, etc.) needed for implantable device use is sealed in pouch made of ethylene oxide porous Tyvek and the entire sealed pouch with an implantable device inside is sterilized with ethylene oxide for 24 hours. In this embodiment, the aptamer sensor is sterilized while sealed in a sterile storage package which is porous to the sterilant used. In this case, the device may remain sterile and shelf stable for, in alternate embodiments, at least 2 weeks, 2 months, or 6 months. The same sterilization of the entire device, or a sensor subcomponent and or storage materials, in a sterile package may also be performed for non-implantable devices. For non-packaged components, the duration of sterilization may be hours or shorter and may utilize elevated temperatures of 50-60° C. or more. Ethylene oxide can damage DNA and therefore aptamers, as it is an alkylation agent and can participate in the transition of pyrimidine bases from C to Tor U and from U to C. Therefore, even with an aptamer storage material such as thin trehalose, dried scrum, polyethylene glycol, polyvinyl alcohol, a dried salt layer such as NaCl, other polymers, dextran, and mixtures thereof may also incorporate at least one ethylene oxide protectant. Useful ethylene oxide protectants include Grinard compounds such as organomagnesium compounds, MgCl2, ferric chloride, copper oxide, diisocyanate compounds such as methylene diphenyl diisocyanate, or other suitable compounds that inhibit ethylene oxide and/or frec radicals associated with ethylene oxides damage to molecules in the sensor such as aptamers. In some embodiments, this may require 0.1's to 10% of concentration of the ethylene oxide protectant in the aptamer storage material or in some cases the aptamer storage material can be the ethylene oxide protectant itself. To enable enough capacity to inhibit ethylene oxide chemistry locally near the aptamers or blocking layer molecules, or the ability for ethylene oxide to diffuse to the aptamers or blocking layer the thickness of the layer containing the ethylene oxide inhibitor may be, in alternative embodiments, at least one of >20 nm, >200 nm, or >1 μm thick. The above discussion applies to ethylene oxide but may also be extended to nitrogen dioxide, and use protectants against nitrogen dioxide such as soda-lime, vanadium oxides, or other suitable protectants, including those that work at lower temperatures unlike techniques used in catalytic converters for exhaust systems. Lastly, in embodiments of the present invention, the ethylene oxide protectant may be immobilized inside the device such that it does not harm or irritate the body (such as particles of the protectant held in by a size selective membrane covering the sensor) or by using biosafe protectants such as MgCl2.

With reference to embodiments of the present invention, the following sterilization examples are disclosed which have the most impact on the aptamer material in most sensing devices. In some devices, blocking layers, membranes, or other materials may be more sensitive material to degradation, but such materials typically can be replaced or altered. Unlike other materials, aptamer materials typically cannot be adequately altered inherently in their chemical makeup, or else their form and function is also altered.

With reference to embodiments of the present invention, gamma irradiation can be used to sterilized a device using 1's to 10's of kGy (in one embodiment, a range of 25-40 kGy), and Xray irridiation at 10's to 100's of Gy. E-beam dosing can be implemented 1's to 10's of kGy (in one embodiment, a range of 25-40 kGy). UV irradiation is possible but surfaces must be exposed to the UV. In one embodiment, an electrochemical aptamer sensor has a sucrose coating. The sucrose coating absorbs the UV and prevents full sterilization if the sucrose is too thick. Likewise, a sensor stored in denatured serum or a portion of solutes from serum could be difficult to UV sterilize due to absorption of UV. Therefore, alternately the aptamer storage material could be thin and less than the size of spores for example as taught above, but include a biocompatible UV-absorber such as those used in sun-screen products to protect the aptamer sensors. Ideally such UV-absorbers are water soluble such as benzophenone or other UV absorbers used in water-soluble sunscreens such that UV-absorber can be dissolved or mixed in a water soluble aptamer storage material such as trehalose. Alternately, alcohol such as ethanol or isopropanol, or other solvents could be used to sterilize a device. In one embodiment, an electrochemical aptamer sensor is coated with a storage material such as glucose, trehalose, or other sugar, then exposed to ethanol, isopropyl, or propanol alcohols or other sterilizing organic solvents in which the sugar or other aptamer storage material is poorly soluble. The storage material thickness could be very thin (10's of nm) or dissolve slowly to a thickness that is so thin, that any microbes or other contaminants that are larger in size would be exposed to the solvent before the aptamer is exposed to the solvent, hence sterilizing without damaging the aptamer sensor. Aqueous sterilization fluids such as glutaraldehyde or thiomersal can also be utilized during sterilization. The above methods can also be deployed during storage to continually sterilize slowly or maintain sterilization (e.g. like techniques used for vaccines).

With reference to embodiments of the present invention, one or more of the sterilization methods above may also be compatible with a device that contains an enzymatic glucose sensor and at least one aptamer sensor, for example for addressing measures of diabetes and diabetic comorbidities such as cardiac disease on the same device. In such cases, the sterilization method must be compatible with both sensor modalities (enzymatic and aptamer).

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method of measuring analytes in a subject in vivo for a period of time comprising:
a) storing a device, the device comprising:
at least one sensor comprising an aptamer material;
at least one feature for coupling said sensor to an analyte in the subject in vivo;
at least one sterile packaging material enabling a storage state;
wherein the at least one sensor is contained in the sterile packaging material, and further, wherein the at least one sensor and the at least one feature are sterile;
b) removing the sterile packaging material from the device; and
c) using the feature to couple the sensor to an analyte in the subject.

2. The method of claim 1 wherein the device further comprises at least one aptamer storage material.

3. The method of claim 1 wherein the at least one sensor is capable of producing a signal when exposed to the analyte; and further, wherein the signal has an initial level of strength prior to storage of the sensor in sterile packaging material.

4. The method of claim 1 wherein the sensor has a second level of strength after storage of the sensor in sterile packaging material, and said second level of strength differs from the initial level of strength such that the analyte is still measurable in-vivo.

5. The method of claim 4 wherein the second level of strength is lower than the initial level of strength by an amount selected from the group consisting of less than 80%, less than 40%, less than 20%, less than 10%, and less than 5%.

6. The method of claim 1 wherein the device remains sterile and shelf stable for a period of time selected from the group consisting of at least 2 weeks, at least 2 months, and at least 6 months.

7. The method of claim 1 wherein the device comprises a plurality of device components.

8. The method of claim 1 further comprising sterilizing the device by sterilizing the device components and then assembling the device components in a sterile environment.

9. The method of claim 1 further comprising sterilizing the device by separately sterilizing the device and the device components and integrating the device and device components in a sterile environment.

10. The method of claim 1 further comprising sterilizing the device using conditions comprising dry heat in an inert environment.

11. The method of claim 1 wherein the device is sterilized using conditions comprising an inert environment at greater than or equal to 120 Celsius for more than 1 hour.

12. The method of claim 2 further comprising sterilizing the device wherein the sterilization comprises sterilizing the sensor and the aptamer storage material together with a packaging material that is stable during sterilization.

13. The method of claim 2 wherein the aptamer storage material has a thickness selected from the group consisting of at least <500 nm, at least <100 nm, and at least <20 nm.

14. The method of claim 1 wherein the device is sealed in a pouch made of ethylene oxide porous Tyvek.

15. The method of claim 1 wherein the sensor is sterilized with a sterilant while it is sealed in the sterile packaging material, and further, wherein the sterile packaging material is porous to the sterilant.

16. The method of claim 15 wherein the device remains sterile and shelf stable for a period of time selected from the group consisting of at least 2 weeks, at least 2 months, and at least 6 months.

17. The method of claim 2 wherein the aptamer storage material comprises an ethylene oxide protectant.

18. The method of claim 2 wherein the aptamer storage material comprises a composition selected from the group consisting of thin trehalose, dried serum, polyethylene glycol, polyvinyl alcohol, a dried salt layer, other polymers, dextran, and mixtures thereof.

19. The method of claim 18 wherein the aptamer storage material further comprises an ethylene oxide protectant.

20. The method of claim 19 wherein the ethylene oxide protectant is comprised in a layer having a thickness selected from the group consisting of >20 nm, >200 nm, and >1 μm.

21. The method of claim 1 wherein an ethylene oxide protectant is immobilized inside the device such that it does not harm or irritate the body.

22. The method of claim 1 wherein an ethylene oxide protectant is held inside the device by a size selective membrane covering the sensor.

23. The method of claim 1 wherein the device is sterilized using a method selected from the group consisting of gamma irradiation, Xray irradiation, E-beam dosing, UV irradiation and combinations thereof.

24. The method of claim 1 wherein the device is sterilized using gamma irradiation at a range from about 25 to about 40 kGy.

25. The method of claim 1 wherein the device is sterilized using Xray irradiation at a range from about 10 to about 500 Gy.

26. The method of claim 1 wherein the device is sterilized using E-beam dosing at a range from about 25 to about 40 kGy.

27. The method of claim 2 wherein the aptamer storage material has a thickness that is less than the size of spores.

28. The method of claim 2 wherein the aptamer storage material comprises a biocompatible UV-absorber.

29. The method of claim 1 wherein the device is sterilized using a solvent.

30. The method of claim 1 wherein the device is sterilized using a solvent selected from the group consisting of ethanol, isopropanol, propanol, and other alcohols.

31. The method of claim 2 wherein the sensor is coated with an aptamer storage material selected from the group consisting of glucose, trehalose, and other sugars; then exposed to a solvent selected from the group consisting of isopropanol, propanol, and other sterilizing organic solvents; wherein the sugar or other aptamer storage material is poorly soluble in the solvent.

32. The method of claim 1 further comprising sterilizing the device using conditions comprising an inert environment.

33. An in-vivo continuous biosensing device comprising:
at least one sensor comprising an aptamer material;
at least one feature for coupling said sensor to an analyte in the human body; and
at least one sterile packaging material enabling a storage state, wherein the at least one sensor is contained in the sterile packaging material;
wherein the at least one sensor and the at least one feature are sterile.

34. The device of claim 33 wherein the device is capable of activating a sterilization state that imparts sterilization on at least one component of the device.

35. The device of claim 33 wherein the aptamer material further comprises redox tags.

36. The device of claim 33 wherein the aptamer material further comprises fluorescent tags.

37. The device of claim 33 further comprising at least one aptamer storage material.

38. The device of claim 33 further comprising a sterilization material that exists in the storage state.

39. The device of claim 37 wherein the storage material is anhydrous.

40. The device of claim 37 wherein the storage material has a thickness that is selected from the group consisting of <500 nm, <100 nm, and <20 nm.

41. The device of claim 37 comprising at least one sterilization chemical and wherein the storage material contains at least one protectant against the sterilization chemical.

42. The device of claim 41 wherein the at least one protectant against the sterilization chemical is either sealed in the device such that it is biosafe during operation of the device or biosafe itself.

43. The device of claim 37 wherein there is at least one sterilization solvent and the storage material is not fully dissolvable in the solvent during sterilization.

44. The device of claim 33 wherein the device additionally contains at least one continuous sensor for glucose.
